(19) United States
(12) Patent Application Publication
(10) Pub. No.: US 2004/0141934 A1
(43) Pub. Date: Jul. 22, 2004

(54) Two-phase roll-on cosmetic product

(75) Inventors: Lin Fei, Kendall Park, NJ (US); Suman Chopra, Dayton, NJ (US); Neeta Patel, Piscataway, NJ (US)

Correspondence Address: Colgate-Palmolive Company, 909 River Road, P.O. Box 1343, Piscataway, NJ, 08855-1343, US

(73) Assignee: Colgate-Palmolive Company

(21) Appl. No.: 10/346,834

(22) Filed: Jan. 17, 2003

(51) Int. Cl. A61K 7/32

(52) U.S. Cl. 424/65

(57) ABSTRACT

A two-phase, elastomer-free, low viscosity, high water roll-on antiperspirant and/or deodorant composition comprising:

(A) a non-polar phase having a viscosity up to 200 centipoise and comprising:

(a) 0.1-40 weight % of a volatile and/or a nonvolatile silicone selected from the group consisting of linear and cyclic organo-substituted polysiloxanes wherein the viscosity is less than 5 centistokes for volatile silicones and in the range of 5-20 centistokes for non-volatile silicones; (b) 0-25 weight % of a straight or branched chain hydrocarbon polymer which has an average molecular weight in the range of 450-6000 daltons; (c) 0-15 weight % of one or more of a selected low viscosity, lipophilic emollient; and

(B) a polar phase having a viscosity in the range of 10-2,000 centipoise and comprising:

(a) at least 5 weight % of an antiperspirant active; (b) an aqueous component comprising at least 40% water and a sufficient amount of a C2-3 alcohol, a glycol or a polyhydric alcohol so that the antiperspirant active is dissolved in the aqueous component; and (c) a selected thickening agent;

wherein the ratio of oil phase to water phase is in the range of 15:85-40:60; and

whereby the composition is able to form a temporarily stabilized emulsion after shaking for a period not exceeding 24 hours.

### FIELD OF THE INVENTION

This invention relates to a two-phase, elastomer-free, low viscosity, high water roll-on cosmetic product, especially for use in the field of antiperspirants and/or deodorants.

### BACKGROUND OF THE INVENTION

Current roll-on products in the market frequently involve emulsions and/or the suspension of an antiperspirant active in the formulation. The products frequently require the use of one or more surfactants to stabilize and/or compatabilize two non-compatible phases. There are two issues associated with the use of surfactants. The first issue is skin irritation. Unless the surfactant system is carefully selected, the use of such materials may cause skin irritation. The second issue is efficacy. It may be the case that in some systems, the use of a surfactant may interfere with the gel plug formation by which many antiperspirant actives work to reduce perspiration.

Another group of benefits may also be achieved with the reduction and/or elimination of surfactants in cosmetic products that contain an antiperspirant active. These benefits include improving the dry feel of the product, reducing the amount of non-white residue on skin and clothing, and reducing the tackiness of the product.

Two-phase systems have been seen in a few cosmetic applications including liquids and solids. One commercial liquid two-phase product from The Dial Corporation (Scottsdale, Ariz.) is Nature's Accents® Bath Treats, a bubble bath product comprising water, cetearyl ethylhexanoate, sodium laureth sulfate, cocamidopropyl betaine, fragrance, glycerin, sodium cocoyl glutamate (and) disodium cocoyl glutamate, PEG-8, DMDM hydantoin, tetrasodium EDTA, hydrolyzed milk protein, honey extract, and coloring agents. As a cleansing material this product is formulated with surfactants. This product has a creamy milky top layer with a clear colored layer under it.

A second commercial liquid two-phase product is Cloud Dance™ breeze cologne spray distributed by CCA Industries, Inc. (East Rutherford, N.J.). This product lists the following ingredients: SD alcohol, 40D, hexamethyldisiloxane, fragrance, distilled water, sodium chloride, coloring agents, and benzophenone-3. This product is clear with two bands of differing colors.

There are patent references that describe two-phase or multiphase technology. U.S. Pat. No. 4,120,948 to Shelton describes a two-phase stick antiperspirant having (1) a substantially anhydrous antiperspirant phase comprising a water-insoluble, high melting point wax, a liquid emollient, and high levels of a particulate, antiperspirant active material and (2) a gel phase comprising a polyhydric alcohol gelled with either a fatty acid soap or a fatty acid amide.

U.S. Pat. No. 4,438,095 to Grollier et al describes a liquid cosmetic composition comprising two separate liquid phases. The first phase is an aqueous phase in which at least one cationic polymer is dissolved. The composition contains no detergent agent or foaming agents.

U.S. Pat. No. 4,767,741 to Komor et al teaches a two-phase liquid cosmetic composition comprising an oil phase, and organic liquid/water phase and insoluble solid particles absorbed on the interface between the two phases, wherein the solid particles are the in situ precipitation product of at least a first salt solution and a second salt solution (added to the oil phase and organic/water phase during blending thereof).

U.S. Pat. No. 4,973,473 to Schneider et al teaches (in a preferred embodiment) a composition comprising two discrete gel phases. The composition includes an emollient complex containing a selected carboxylic acid amide(s), a mucopolysaccharide, at least one skin structuring protein and an astringent.

U.S. Pat. No. 4,980,155 to Shah et al teaches a two-phase cosmetic composition comprising a color phase which includes a first phase comprising a film forming agent, at least one colorant, an emulsifier and water; and a second phase comprising a gel phase comprising a water soluble polymer and water. The phases are miscible with each other but are disposed in discrete side by side separate phases.

U.S. Pat. No. 5,213,799 to Foring et al describes skin treating compositions which comprise a transparent oil phase and a transparent aqueous phase, preferably with humectant effect, which permits a homogenous mixture when shaken together and thereafter separates again into two transparent phases. The cosmetic composition includes the use of 0.1-1.0 weight % of C12-C18 fatty acid triglycerol ester.

U.S. Pat. No. 5,290,555 to Guthauser et al teaches compositions with structural color in which two phases are selected with the same refractive index but different dispersive power. Either or both of the phases may contain cosmetically active ingredients.

U.S. Pat. No. 5,474,777 to Marion et al describes liquid cleansing compositions consisting of an oily phase and an aqueous phase, wherein the oily phase consists of at least one dialkylphosphate and, optionally, products miscible therein, and the aqueous phase contains one or more ionic surfactants.

U.S. Pat. No. 5,593,663 to Leng et al describes antiperspirant actives which are amphiphilic materials that, upon contact with perspiration, form a water-insoluble liquid crystal phase of greater than one-dimensional periodicity.

U.S. Pat. No. 5,654,362 to Schultz et al describes silicone oils and solvents thickened by silicone elastomers and suitable for use in products such as antiperspirants and deodorants.

U.S. Pat. No. 5,919,437 to Lee et al describes solid cosmetic compositions including cosmetic cream compositions containing silicone elastomers as gelling agents.

U.S. Pat. No. 6,019,991 to Tanaka et al teaches a two-phase cosmetic composition comprising separate and distinct oil and aqueous phases which form a highly temporary clear emulsion when shaken together. A clear package is used. One phase may be colored while the other is usually water white.

U.S. Pat. No. 6,180,587 to Fuller et al teaches a multiple phase composition comprising a lower aqueous phase comprising at least 1 weight % of a polymer or copolymer selected from the group consisting of polyacrylate, polystyrene sulfonate, polyvinyl-pyrrolidone, maleic anhydride and mixtures thereof and an upper aqueous phase having a cleansingly effective amount of a surfactant.

PCT case WO 00/67712 discloses a solid stick containing antiperspirant actives and formed with a core phase and an outer phase.

Conventional roll-on products used as antiperspirants and/or deodorants are usually emulsions or anhydrous suspensions. Emulsions can be oil-in-water or water-in-oil systems wherein one phase is dispersed in the other. In emulsions both phases remain together because of the addition of surfactants. Emulsion products tend to have a wet feel, a higher level of tackiness and some irritation issues. Anhydrous suspensions are composed of powders suspended in anhydrous liquids. They typically include a relatively high percentage of suspending agents that contribute to white residues being left on skin and clothing. While the anhydrous suspension do not produce a wet feel, they exhibit other problems such as settling of the powder over time and leakage of the carrier liquid, especially if the product is not continuously shaken.

The present invention reduces and/or eliminates some of the aforementioned problems. In particular, the compositions of the present invention are able to provide two phase antiperspirants and/or deodorants which have improved efficacy, reduced irritation on skin, reduced white residue on skin and clothing, and improved aesthetics such as reducing the tackiness.

### BRIEF SUMMARY OF THE INVENTION

This invention is a two phase, elastomer-free, low viscosity, high water roll-on cosmetic product, especially for use in the field of antiperspirants and/or deodorants. The roll-on product is made with a polar phase and a non-polar phase wherein a cosmetically active ingredient effective as an antiperspirant and/or a deodorant is dissolved in one of the phases.

The composition is packaged in a conventional roll-on dispenser. Before the product is applied the container is shaken or agitated forcefully enough to temporarily mix the two phases. The product is applied while the two phases are mixed. The following description includes both a formulation which comprises the ingredients listed and a composition which is made by combining the ingredients listed. Note that for the units used, centistoke=centipoise/density. Thus, when the density is close to 1, the centistoke and centipoise measurements will be about the same.

The non-polar phase can be clear, translucent or opaque, has a viscosity up to 200 centipoise (preferably less than 20 centipoise), and is made by combining the following ingredients:

(a) 0.1-40 weight % (more particularly 5-30%) of a volatile and/or a nonvolatile silicone selected from the group consisting of linear and cyclic organo-substituted polysiloxanes wherein the viscosity is less than 5 centistokes for volatile silicones and in the range of 5-20 centistokes for non-volatile silicones;

(b) 0-25 weight % (more particularly 1-10%) of a straight or branched chain hydrocarbon polymer which has an average molecular weight in the range of 450-6000 daltons;

(c) 0-15 weight % (more particularly, 1-10% and, even more particularly, 1-5%) of one or more low viscosity, lipophilic emollients selected from the group consisting of:

(i) 0-10 weight % (with a particular range being 1-8%) of a non-polar hydrocarbon (preferably branched such as with an “iso” group) having from 4-30 carbons (preferably 4-20 carbons and, more preferably, 6-20 carbons);

(ii) 0-10 weight % (with a particular range being 1-8%) of a benzoic acid ester selected from the group consisting of C12-C20 benzoate esters (for example, a C12-15 alkyl benzoate such as FINSOLV TN);

(iii) 0-8 weight % (with a particular range being 4-6% and especially 5%) of a propoxylated fatty alcohol having 4-16 carbons and 2-14 moles of propoxylation (for example, PPG-3 myristyl ether);

(d) optionally one or more ingredients selected from the group consisting of

(i) 0-0.2 weight % (particularly 0.001-0.01 weight %) of a silicone surfactant having an hydrophilic lipophilic balance (“HLB value”)≦13 (so as to temporarily stabilize the emulsion for ease of application, for example for a time not exceeding 24 hours, particularly not exceeding 15 hours (noting that compositions can be made whose stability is in a range as low as 1-20 minutes);

(ii) 0-2 weight % (particularly 0.1-1%) hydrophobically treated amorphous colloidal fumed silica;

(iii) 0- 2 weight % fragrance (particularly 0.5 to 1 weight %);

(iv) 0-1.00 weight % vitamins (particularly 0.01-1.00 weight %) (especially vitamin E or a precursor); and

(v) 0.0-0.002 weight % coloring agent (for example, a cosmetic pigment).

The polar phase can be clear, translucent or opaque. In general it may contain one or more members selected from water and or a polyhydric alcohol wherein the cosmetically active ingredients are dissolved in this polar phase. The polar phase should also have a viscosity in the range of 10-2,000 centipoise (“cps”). The polar phase comprises:

(a) at least 5 weight % of an antiperspirant active;

(b) an aqueous component comprising at least 40% water and a sufficient amount of a C2-3 alcohol, a glycol or a polyhydric alcohol so that the antiperspirant active is dissolved in the aqueous component (for example, 40-70% water, and up to 20.00 weight % (maximum) of one or more of ethyl alcohol, isopropyl alcohol, a glycol selected from the group consisting of propylene glycol, dipropylene glycol, tripropylene glycol and methylpropylene glycol, and mixtures of any of the foregoing);

(c) a thickening agent selected from the group consisting of:

(i) 0.05-2.5 weight % of a water soluble cationic derivative selected from the group consisting of hydroxyethyl cellulose and its copolymers (preferably Polyquaternium-10 (Celquat SC 240 C from National Starch, Finderne, N.J.)), and hydroxypropyl cellulose and its copolymers, provided that the viscosity of the polar phase does not exceed 2,000 cps; and

(ii) 0.1-5 weight % of a starch modified polymer which is a sodium hydroxypropyl starch phosphate (for example, Pure Gel® products from Grain Processing Corporation, Muscatine, Iowa, such as those sold under the B990, B992, B994, and B980 designations);

(d) optionally one or more ingredients selected from the group consisting of

(i) micas (≦1.0 weight %, with an average particle size in the range of 10-125 microns and preferably less than 30 microns);

(ii) suspending agents (for example, 1-3 weight % of Bentone 38 with the addition of a polar additive, for example 0.3-1.0 weight % of propylene carbonate);

(iii) antimicrobial agents (which may also be considered as a cosmetic ingredient if used in sufficient amount to inhibit bacteria growth under the arm), for example, a member of the group consisting of bacteriostatic quaternary ammonium compounds such as 2-amino-2-methyl-1-propanol (AMP), cetyl-trimethylammonium bromide, cetyl pyridinium chloride, 2, 4, 4′-trichloro-2′-hydroxydiphenylether (Triclosan), N-(4-chlorophenyl)-N′-(3,4-dichlorophenyl)urea (Triclocarban), silver halides, octoxyglycerin (Sensiva™ SC 50); 3,7,11,-trimethyldodeca-2,6,10-trienol (Farnesol from Dragoco, Totowa, N.J.); and various zinc salts (for example, zinc ricinoleate). The bacteriostat can, illustratively, be included in the composition in an amount of 0-5%, particularly 0.01-1.0% by weight, of the total weight of the composition (Triclosan, can illustratively be included in an amount of from 0.05% to about 0.5% by weight, of the total weight of the composition.);

(iv) masking agents in an amount of 0-5.0 weight % (particularly 0.05-2%) by weight based on the total weight of the composition particularly if an unscented product is desired;

(v) 0-5 weight % of one or more polymers of ethylene oxide, for example PEG-4, PEG-6, PEG-8, PEG-9, PEG-10, PEG-12, PEG 14, Carbowax PEG-200, Carbowax PEG-300, Carbowax PEG-400, Carbowax PEG-600, particularly 0.1-1.0 weight % of PEG-12; and

(vi) 0-0.5 weight % of a water soluble surfactant having an hydrophilic lipophilic balance (“HLB value”)≧10 (for example, a member of the group consisting of Ceteth-20 sucrose stearate, Oleath-10 (sold under the tradename Volpo 10 from Croda, Inc., N.J.), Oleath-20 (sold under the trade name Volpo 20), PEG 7 glycerol cocoate, and sorbitan 20 ethylene oxide monolaurate); wherein the ratio of oil phase to water phase is in the range of 15:85-40:60; and whereby the composition is able to form a temporarily stabilized emulsion when shaken with a period of stability not exceeding 24 hours.

### DETAILED DESCRIPTION OF THE INVENTION

For the volatile silicones used in this invention, linear or cyclic materials may be used alone or in combination. Linear volatile methyl siloxanes (“VMS”) have the formula (CH₃)₃ SiO{(CH₃)₂ SiO}_{y} Si(CH₃)₃. The value of y is 0-5. Cyclic VMS have the formula {(CH₃)₂ SiO}_{z}. The value of z is 3-6. Preferably, these volatile methyl siloxanes are linear or cyclic with a viscosity in the range of 0.5-5 centistokes (with the “cst” unit being the same as mm²/s).

Representative linear volatile methyl siloxanes (I) are hexamethyldisiloxane (MM) with a boiling point of 100 degrees C., viscosity of 0.65 mm²/s, and formula Me₃ SiOSiMe₃ ; octamethyltrisiloxane (MDM) with a boiling point of 152 degrees C., viscosity of 1.04 mm²/s, and formula Me₃ SiOMe₂ SiOSiMe₃; decamethyltetrasiloxane (MD₂ M) with a boiling point of 194 degrees C., viscosity of 1.53 mm²/s, and formula Me₃ SiO(Me₂ SiO)₂ SiMe₃; dodecamethylpentasiloxane (MD₃ M) with a boiling point of 229 degrees C., viscosity of 2.06 mm²/s, and formula Me₃ SiO(Me₂ SiO)₃ SiMe₃; tetradecamethylhexasiloxane (MD₄M) with a boiling point of 245 degrees C., viscosity of 2.63 mm²/s, and formula Me₃ SiO(Me₂ SiO)₄ SiMe₃; and hexadecamethylheptasiloxane (MD₅ M) with a boiling point of 270 degrees C., viscosity of 3.24 mm²/s, and formula Me₃ SiO(Me₂ SiO)₅ SiMe₃.

Representative cyclic volatile methyl siloxanes (II) are hexamethylcyclotrisiloxane (D₃) a solid with a boiling point of 134 degrees C. and formula {(Me₂)SiO}₃; octamethylcyclotetrasiloxane (D₄) with a boiling point of 176 degrees C., viscosity of 2.3 mm²/s, and formula {(Me₂)SiO}₄; decamethylcyclopentasiloxane (D₅) with a boiling point of 210 degrees C., viscosity of 3.87 mm²/s, and formula {(Me₂)SiO}₅; and dodecamethylcyclohexasiloxane (D₆) with a boiling point of 245 degrees C., viscosity of 6.62 mm²/s, and formula {(Me₂)SiO}₆ (with a particular group of cyclics including D5 and D6 cyclomethicones).

Particular examples of suitable volatile silicones include DC-244 Fluid, DC-245 Fluid, DC 246 Fluid, DC-344 Fluid, DC-345 Fluid, DC 200 Fluid (with 0.65 cst viscosity) to DC 200 Fluid (with 5 cst viscosity), and DC-1184 Fluid (a mixture of low molecular weight volatile and non-volatile silicones most of which are linear and volatile, such material has a boiling point greater than 35 degrees and a viscosity of about 1.6 centistokes) all of which are from Dow Corning Corp.), and especially decamethylcyclopentasiloxane (DC-245 Fluid).

Silicones are linear organo-substituted polysiloxanes which are polymers of silicon/oxygen with general structure:

(1) (R²⁰)₃SiO(Si(R²¹)₂O)ₓSi(R²²)₃ where R²⁰, R²¹ and R²² can be the same or different and are each independently selected from the group consisting of phenyl and C1-C60 alkyl; or

(2) HO(R²⁴)₂SiO(Si(R²⁵)₂O)ₓSi(R²⁶)₂OH, where R²⁴, R²⁵ and R²⁶ can be the same or different and are each independently selected from the group consisting of phenyl and C1-C60 alkyl;

(with specific examples including dimethicone, dimethiconol behenate, C₃₀₋₄₅ alkyl methicone, stearoxytrimethylsilane, phenyl trimethicone and stearyl dimethicone).

In general, silicone copolyols useful in the present invention include copolyols of the following Formulae I and II. Formula I is:

(R¹⁰)₃—SiO—[(R¹¹)₂—SiO]ₓ—[Si(R¹²)(R^{b}—O—(C₂H₄O)ₚ—(C₃H₆O)ₛ—R^{c})O]_{y}—Si—(R¹³)₃ Formula I

wherein each of R¹⁰, R¹¹, R¹², and R¹³ may be the same or different and each is selected from the group consisting of C1-C6 alkyl; R^{b} is the radical —CₘH₂ₘ—; R^{c} is a terminating radical which can be hydrogen, a C1-C6 alkyl group, an ester group such as acyl, or an aryl group such as phenyl; m has a value in the range of 2-8; p and s have values such that the oxyalkylene segment —(C₂H₄O)ₚ—(C₃H₆O)ₛ— has a molecular weight in the range of 200-5,000; the segment preferably having 50-100 mole percent of oxyethylene units, —(C₂H₄O)ₚ—, and 1-50 mole percent of oxypropylene units, —(C₃H₆O)ₛ—; x has a value of 8-400; and y has a value of 2-40. Preferably each of R¹⁰, R¹¹, R¹², and R¹³ is a methyl group; R^{c} is hydrogen; m is 3 or 4 whereby the group R^{b} is most preferably the radical —(CH₂)₃—; and the values of p and s are such as to provide a molecular weight of the oxyalkylene segment, —(C₂H₄O)ₚ—(C₃H₆O)ₛ—, in the range of 1,000-3,000. Most preferably p and s are selected to have a value in the range of 18-28. Formula II is

(R¹⁰)₃—SiO—[(R¹¹)₂—SiO]_{w}—[Si(R¹²)(R^{b}—O—(C₂H₄O)ᵣ—R^{c})O]_{z}—Si—(R¹³)₃ Formula II

wherein r has a value in the range of 6-16; w has a value in the range of 6-100; z has a value in the range of 1-20, and the other moieties have the same definition as described for Formula I.

It should be understood that in both Formula I and II shown above, the siloxane-oxyalkylene copolymers of the present invention may, in alternate embodiments, take the form of endblocked polyethers in which the linking group (R^{b}), the oxyalkylene segments, and the terminating radical (R^{c}) occupy positions bonded to the ends of the siloxane chain, rather than being bonded to a silicon atom in the siloxane chain. Thus, one or more of the R¹⁰, R¹¹, R¹², and R¹³ substituents which are attached to the two terminal silicon atoms at the end of the siloxane chain can be substituted with the segment —(R^{b}—O—(C₂H₄O)ₚ—(C₃H₆O)ₛ—R^{c}) or with the segment —(R^{b}—O—(C₂H₄O)ᵣ—R^{c}). In some instances it may be desirable to provide the segment —(R^{b}—O—(C₂H₄O)ₚ—(C₃H₆O)ₛ—R^{c}) or —(R^{b}—O—(C₂H₄O)ᵣ—R^{c}) at locations which are in the siloxane chain as well as at locations at one or both of the siloxane chain ends.

Particular examples of suitable dimethicone copolyols which are available either commercially or experimentally from a variety of suppliers include DOW CORNING® 5225C (from Dow Corning Corporation, Midland, Mich.) which is a 10% dimethicone copolyol in cyclomethicone; DOW CORNING® 2-5185C which is a 45-49% dimethicone copolyol in cyclomethicone; SILWET L-7622 (from Witco Corporation, Greenwich, Conn.); ABIL EM97 (from Goldschmidt Chemical Corporation, Hopewell, Va.) which is a 85% dimethicone copolyol in D5 cyclomethicone; and FPD 4668 (from Shin-Etsu Corporation, Japan which is a fluorine containing organopolysiloxane.

It should also be noted that various concentrations of dimethicone copolyols in cyclomethicone can be used. While a concentration of 10% in cyclomethicone (which can be D4, D5, D6 or mixtures thereof) is frequently commercially available, other concentrations can be obtained such as, for example, by stripping off the cyclomethicone or adding additional cyclomethicone. The higher concentration materials such as DOW CORNING® 2-5185C is of particular interest.

In one particular embodiment 0.1-2% (particularly 0.1-0.5%) of a 10% silicone copolyol such as dimethicone copolyol in cyclomethicone (particularly a D5 or D6 cyclomethicone) mixture may be used, wherein the amount of the mixture added is selected so that the level of silicone copolyol in the cosmetic composition is in the range of 0 to about 0.2 weight % (particularly 0 to about 0.05 %) (for example, 0.25-0.4% of a 40-50% dimethicone copolyol in cyclomethicone mixture).

Hydrocarbons as used in this application are a group of compounds derived from petroleum and contain only carbon and hydrogen. Their structures can vary widely and include aliphatic, alicyclic and aromatic compounds. Specific examples include paraffin, petrolatum, hydrogenated polyisobutene, polybutene and mineral oil. Hydrocarbons of particular interest for use in this invention include isoparaffinic fluids having 4-30 carbons (especially 7-20 carbons) such as C7-8 isoparaffin, C8-9 isoparaffin, C10-11 isoparaffin, C11-12 isoparaffin, C11-13 isoparaffin, C13-14 isoparaffin, C12-20 isoparaffin, especially C11-12 isoparaffin (for example, Isopar H from Exxon Chemical Company, Baytown, Tex.), and other branched chain hydrocarbons such as isododecane (Permethyl 99A), isoeicosane (Permethyl 102A), isohexadecane (Permethyl 101A) (the Permethyls being available from Preperse, Inc., South Plainfield, N.J.), and combinations of any of the foregoing.

Particular examples of benzoate esters which can be used in this invention include isostearyl benzoate, PPG-15 stearyl ether benzoate, octyldodecyl benzoate, and C12-15 alkyl benzoate and those described in U.S. Pat. No. 4,791,097 and U.S. Pat. No. 5,270,461, incorporated by reference herein with respect to the description of such esters. These include compositions of formula: PHENYL —C(O)OR³⁰ where R³⁰ is:

(a) a branched or linear alkyl of 20-28 carbons; or

(b) —CH(CH₃)—CH₂(O—CH(CH₃)CH₂)ₙ—O—R³¹, wherein n is 9-16 and R³¹ is a branched or linear alkyl of 3-22 carbons.

More particular examples of such benzoate esters include isostearyl benzoate, PPG-15 stearyl ether benzoate, octyldodecyl benzoate, and C12-15 alkyl benzoate (for example and preferably, FINSOLV TN from Finetex, Inc. (Elmwood Park, N.J.).

Another particular group of such esters include those marketed by Finetex under the designations FINSOLV® TN (C12-15 alkyl benzoate), FINSOLV® SB (isostearyl benzoate), FINSOLV® P (PPG-15 stearyl ether benzoate), FINSOLV® BOD (octyl dodecyl benzoate), FINSOLV® 116 (stearyl benzoate), FINSOLV® PL-62 (Poloxamer 182 benzoate) and FINSOLV® PL-355 (poloxamer 105 benzoate).

The antiperspirant active can be selected from the group consisting of any of the known antiperspirant active materials. These include, by way of example (and not of a limiting nature), aluminum chlorohydrate, aluminum chloride, aluminum dichlorohydrate, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex PG, aluminum chlorohydrex PEG, aluminum dichlorohydrex PG, and aluminum dichlorohydrex PEG. The aluminum-containing materials can be commonly referred to as antiperspirant active aluminum salts. Generally, the foregoing metal antiperspirant active materials are antiperspirant active metal salts. In the embodiments which are antiperspirant compositions according to the present invention, such compositions need not include aluminum-containing metal salts, and can include other antiperspirant active materials, including other antiperspirant active metal salts. Generally, Category I active antiperspirant ingredients listed in the Food and Drug Administration's Monograph on antiperspirant drugs for over-the-counter human use can be used. In addition, any new drug, not listed in the Monograph, such as aluminum nitratohydrate and its combination with zirconyl hydroxychlorides and nitrides, or aluminum-stannous chlorohydrates, can be incorporated as an antiperspirant active ingredient in antiperspirant compositions according to the present invention.

Particular types of antiperspirant actives include aluminum zirconium trichlorohydrex and aluminum zirconium tetrachlorohydrex either with or without glycine. A particular antiperspirant active is aluminum zirconium glycine salts with enhanced efficacy due to improved distribution of the molecular species such as described in PCT publication number WO 92/19221, and/or solutions of such salts made with water and/or propylene glycol.

Antiperspirant actives can be incorporated into compositions according to the present invention in amounts in the range of 0.1-25% of the final composition, but the amount used will depend on the formulation of the composition. For example, at amounts in the lower end of the broader range (for example, 0.1-10% on an actives basis), a deodorant effect may be observed. At lower levels the antiperspirant active material will not substantially reduce the flow of perspiration, but will reduce malodor, for example, by acting as an antimicrobial material. At amounts of 10-25% (on an actives basis) such as 15-25%, by weight, of the total weight of the composition, an antiperspirant effect may be observed.

The glycol or polyglycol is selected from the group consisting of ethylene glycol, propylene glycol, 1,2-propanediol, diethylene glycol, triethylene glycol, tetraethylene glycol, dipropylene glycol, tripropylene glycol, methyl propanediol, 1,6-hexanediol, 1,3-butanediol, 1,4-butanediol, PEG-4 through PEG-100, PPG-9 through PPG-34, pentylene glycol, neopentyl glycol, trimethylpropanediol, 1,4-cyclohexanedimethanol, 2,2-dimethyl-1,3-propanediol, 2,2,4,4-tetramethyl-1,3-cyclobutanediol, and mixtures thereof. More particular examples of the glycol component include one or more members of the group consisting of propylene glycol, dipropylene glycol, tripropylene glycol, 2-methyl-1,3-propanediol, methyl propylene glycol, low molecular weight (less than 600) polyethylene glycol, low molecular weight (less than 600) polypropylene glycols, and mixtures of any of the foregoing. Propylene glycol is of particular interest because the antiperspirant active is more soluble in this type of glycol. Tripropylene glycol has lower irritancy, but the antiperspirant active is not as soluble in this glycol. Mixtures of glycols may be used to balance these desirable properties.

Compositions according to the present invention can be made by first preparing both phases separately and then combining them in the final container:

Non-polar phase—The linear or cyclic silicones and other non-polar ingredients are blended at room temperature in a vessel. Optionally, fragrances can be added at the end.

Polar phase—In general, all of the ingredients except Polymer JR (Polyquaternium-10) are mixed at 300-400 rpm in a separate beaker to make a clear product. Polymer JR is gradually added to the polar phase with stirring. The mixture is then heated to about 40-50 degrees C. after the addition of Polymer JR. Stirring is continued until the polar phase is clear. The heat is turned off and the polar phase is allowed to cool to room temperature.

After cooling of the polar phase is completed, the non-polar phase is added on top of the polar phase.

If a coloring agent is added, it is first dissolved in a selected amount of polar material such as water, or in a non-polar material such as PPG-3 myristyl ether, C12-15 alkyl benzoates (FINSOLV TN from Finetex, Inc.), and low molecular weight organo-substituted polysiloxanes such as polydimethylsiloxane (for example, DC 200 Fluid having a viscosity of 5 centistokes (“cst”); DC 245 Fluid having a viscosity of 3.87 cst, both of which are available from Dow Corning Corp.) and then added to the appropriate phase.

Each phase can be introduced into dispensing containers known to those skilled in the art for roll-ons. The product from the reservoir comes to the top rolling surface of the dispensing container, and from there may be applied to the skin in the axillary regions to deposit sufficient amounts of antiperspirant and/or deodorant active material to reduce body malodor and/or reduce perspiration in axillary regions of the human body.

The components of the conventional roll-on containers can be made of various materials and can have different shapes. The material of the container can be polypropylene, polyethylene terephthalate (PET), high-density polyethylene or glass. The applicator is usually a hollow ball made of polypropylene. The diameter can vary from 10.4 to 35.5 mm, depending on the design of the container. The ball can be assembled directly in the container or with a special insert (ball housing) depending also on the design of the container. The caps can be of different designs (usually made of polypropylene) with smooth or ribbed walls.

Examples of suitable roll-on dispensers include those described in U.S. Design Pat. No. 402,550 to Poisson; U.S. Pat. No. 6,132,126 to Sheffer et al (an adjustable applicator); U.S. Pat. No. 4,030,844 to Lench et al; U.S. Pat. No. 4,021,125 to Berghahn et al; U.S. Pat. No. 4,033,700 to Spatz; U.S. Pat. No. 5,553,957 to Dornbusch et al; WO 00/64302 to Hindustan Lever Ltd.; and WO 01/03541 to Chang; all of which are incorporated by reference herein to the extent they describe roll-on dispensers. These references also include the use of fumed silica.

The invention may be seen in various forms. For example, one form of the invention has a low viscosity (less than 20 cps) non-polar phase and is free of fumed silica and clays. Alternatively, another choice for this first embodiment is a composition free of clay but which can contain silica. A second embodiment has a non-polar phase made with (a) 5-30% of a cyclomethicone or 5-25% cyclomethicone and up to 5% dimethicone (wherein the dimethicone has a viscosity of less than 5 cst); and (b) 1-10% of a low viscosity, lipophilic emollient (especially C12-15 alkyl benzoate or PPG-3 myristyl ether). A third embodiment has a non-polar phase made with (a) 5-20% of a cyclomethicone and up to 10% dimethicone (wherein the dimethicone has a viscosity of less than 5 cst); and (b) 1-10% of a low viscosity, lipophilic emollient (especially C12-15 alkyl benzoate or PPG-3 myristyl ether). A fourth embodiment has a non-polar phase made with (a) 5-30% of a cyclomethicone or 5-25% cyclomethicone and 5% dimethicone (wherein the dimethicone has a viscosity of less than 5 cst); (b) 1-8% of a low viscosity, lipophilic emollient (especially C12-15 alkyl benzoate or PPG-3 myristyl ether or a non-polar C6-20 hydrocarbon (especially one with an “iso”group)). A fifth embodiment is the same as the fourth embodiment and additionally is free of silica and clay. A sixth embodiment can be made with any of the foregoing embodiments in the non-polar phase and a polar phase made with 1-10% anhydrous ethanol and up to 10% of a glycol as defined above (especially propylene glycol).

Various forms of the invention can be exemplified by the following formulations but these should not be construed as limitations on the invention. Note that all the formulations have the viscosities as noted.

Formulation 1-L

Non-Polar Phase (Viscosity Less Than 20 cps)

10-25weight % pseudo silicone (mixture of mineral oil, dodecanoic acid, dodecyl ester, hexadecanoic acid, octadecyl ester, docosanoic acid octadecyl ester from Strahl & Pitsch Inc., West Babylon, N.Y.)

1-2 weight % cyclomethicone (Dow Corning Fluid 245)

1-3 weight % polybutene (Amoco Corp.)

1-10 weight % PPG-3 myristyl ether (Croda Oleochemicals Inc.)

optionally 0.5-1.0 weight % fragrance.

polar Phase

45-58 weight % of Al Zr tetrachlorohydrex gly (30% in water (Z-522 from Summit))

0.1-0.5 weight % Polyquaternium-10 (Celquat SC 240 C)

2-10 weight % water

optionally 1-10 weight % ethanol

Formulation 2-M

Non-Polar Phase (Viscosity Less Than 200 cps)

0.5-1 weight % Cab-O-Sil ( hydrophobically modified amorphous colloidal silica, Cabot Corp.)

2-20 weight % cyclomethicone (DC 245 Fluid)

10-26 weight % dimethicone from Dow Corning Corp.

optionally 0.5-1.0 weight % fragrance

Polar Phase

45-58 weight % of Al Zr tetrachlorohydrex gly (30% in water (Z-522 from Summit))

0.1-0.5 weight % Polyquaternium-10 (Celquat SC 240 C)

2-10 weight % water

optionally 1-10 weight % ethanol

Formulation 3-AA

Non-Polar Phase (Viscosity Less Than 20 cps)

10-20 weight % cyclomethicone

0.05-0.5 weight % nonionic surfactant (e.g. Emulsogen SRO from Clariant Co., Wayne, N.J.)

0.5-1.0 weight % fragrance

0-5 weight % emollient (such as PPG-3 myristyl ether from Goldschmidt, Hopewell, Va.)

Polar Phase

50-70 weight % (29% active in water) antiperspirant active

10-15 weight % alcohol/glycol (1-10% anhydrous ethanol+0-10% propylene glycol)

1-10 weight % water

0.1-0.3 weight % thickener Polymer JR (Polyquaternium-10 from Amerchol, Edison, N.J.)

Formulation 4-BB

Non-Polar Phase (Viscosity Less Than 20 cps)

20-30 weight % cyclomethicone (DC Fluid 245 from Dow Corning, Midland, Mich.)

0.05-0.2 weight % fluorinated silicone surfactant (For example, FPD 4668 from Shin-Etsu Co., Japan)

0.5-1.0 weight % fragrance

0-5 weight % emollient (such as PPG-3 myristyl ether)

Polar Phase

50-60 weight % (29% active in water) antiperspirant active

5-15 weight % alcohol/glycol (1-10% anhydrous alcohol+0-10% propylene glycol)

1-10 weight % water

0.1-0.3 weight % thickener (Polymer JR)

Formulation 5-CC

Non-Polar Phase (Viscosity Less than 20 cps)

20-35 weight % cyclomethicone/dimethicone (10-35% D5 cyclomethicone+0-10% dimethicone (<5 cst))

0.01-0.05 weight % silicone surfactant (For example 5225C, 10% in cyclomethicone from Dow Corning)

0.5-1.0 weight % fragrance

0-5 weight % emollient (such as FINSOLV TN from Finetex Inc., Elmwood Park, N.J.)

Polar Phase

50-60 weight % (29% active in water) antiperspirant active

5-15 weight % alcohol/glycol (1-10% anhydrous ethanol+0-10% propylene glycol

1-10 weight % water

0.1-0.3 weight % thickener (Polymer JR)

Formulation 6-FF

Non-Polar Phase (Viscosity Less Than 20 cps)

10-15 weight % cyclomethicone/dimethicone (5-15% D5 cyclomethicone+0-10% dimethicone (<5 cst))

0.01-0.05 weight % silicone surfactant (For example 5225C, 10% in cyclomethicone from Dow Corning)

0.5-1.0 weight % fragrance

0-5 weight % emollient (such as FINSOLV TN)

Polar Phase

60-70 weight % (29% active in water) antiperspirant active

10-20 weight % alcohol/glycol (2-10% anhydrous ethanol+0-10% propylene glycol)

1-10 weight % water

0.1-0.5 weight % thickener (Polymer JR)

Formulation 7-GG

Non-Polar Phase (Viscosity Less Than 20 cps)

10-20 weight % cyclomethicone/dimethicone (5-10% D5 cyclomethicone+0-10% dimethicone (<5 cst))

0.5-1.0 weight % fragrance

1-10 weight % emollient (such as PPG-3 myristyl ether)

Polar Phase

50-70 weight % (29% active in water) antiperspirant active

10-20 weight % alcohol/glycol (2-10% anhydrous ethanol+0-10% propylene glycol)

1-10 weight % water

0.1-0.5 weight % thickener (Polymer JR)

Formulation 8-HH

Non-Polar Phase (Viscosity Less Than 20 cps)

20-30 weight % cyclomethicone/dimethicone (10-30% D5 cyclomethicone+0-10% dimethicone (<5 cst))

0.05-0.2 weight % silicone surfactant (For example 5225C, 10% in cyclomethicone from Dow Corning)

0.5-1.5 weight % fragrance

0-5 weight % emollient (such as FINSOLV TN)

Polar Phase

50-60 weight % (29% active in water) antiperspirant active

5-15 weight % alcohol/glycol (0-10% anhydrous ethanol+3-10% propylene glycol)

1-10 weight % water

1-4 weight % thickener (Puregel 990, from Grain Processing Co., Iowa)

### EXAMPLES

The following Examples are offered as illustrative of the invention and are not to be construed as limitations thereon. In the Examples and elsewhere in the description of the invention, chemical symbols and terminology have their usual and customary meanings. Numbers for viscosities and molecular weights are averages. In the Examples as elsewhere in this application (a) values for n, m, etc. in formulas, molecular weights and degree of ethoxylation or propoxylation are averages; (b) temperatures are in degrees C. unless otherwise indicated; and (c) the amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997). Mixing techniques used to make the compositions are those conventionally used in the art including those described above.

### Examples 1-9

### General Method of Making Compositions

Compositions according to the present invention as seen in Examples 1-9 may be made as follows using the amounts and types of ingredients listed under the appropriate Example number. Total percent for each Example is 100%.

Non-polar phase—All of the ingredients for Part A are combined in a beaker and the mixture is stirred at 300-400 rpm using a Lightnin Mixer Model L 1003 until a homogeneous solution is obtained.

Polar phase—The ingredients for the polar phase are combined with stirring. If the polar phase contains a modified starch (such as hydroxypropyl starch phosphate), it must be pre-dispersed in a small part of the antiperspirant active solution. The pre-dispersed material is then added to the rest of the antiperspirant active solution with agitation and the mixture is heated to be at 85-95 degrees C. If the polar phase contains Polyquaternium-10, this is pre-dispersed in the active to form a homogeneous dispersion free of lumps. The mixture is then heated to a temperature in the range of 40-50 degrees C. with agitation until a clear solution is obtained. If the final formulation contains a colorant it must be dissolved in the minimum amount of water and then added in the polar phase.

Combination of the Phases in a Container

Each phase is poured into the container in the ratios indicated in the final composition. There is no special order of addition into the container. The polar phase will tend to go to the bottom.

The method described above can be used to make the following compositions wherein all amounts are weight % based on the total weight of the composition. For these Examples, cyclomethicone is DC 245 from Dow Corning; the surfactant is either a fluorinated silicone surfactant Shin-Etsu FPD-4668 from Shin-Etsu Silicones of America (Akron, Ohio) or Dimethicone copolyol/cyclomethicone 5225C (10%) from Dow Corning; the emulsifier is a sorbitol ester of rapeseed oil (Emulsogen SRO from Clariant Corp., Germany); the silicone fluid is a dimethyl polysiloxane such as DC200 Fluid, 200 cst from Dow Corning, Midland Mich.; the hydrogenated polyisobutene 250 is a branched hydrocarbon from Fanning Corp., Ill.; the antiperspirant active is Al—Zr tetrachlorohydrex gly with a metal:chloride ratio in the range of 0.9:1-1.2:1 as described in U.S. Pat. No. 6,375,937 (obtained from Summit Research Laboratories, Huguenot, N.Y.); a polymeric quaternium ammonium salt of hydroxyethyl cellulose reacted with a trimethyl ammonium substituted epoxide (Polymer JR from Amerchol); a modified starch based polymer, sodium hydroxypropyl starch phosphate (PureGel 990 from Grain Processing Corp.); and Oleath-10 (Volpo 10 from Croda, Inc., Parsippany, N.J.).

TABLE A

| | Example | | | | | | | | |
|---|---|---|---|---|---|---|---|---|---|
| | 1 | 2 | 3 | 4 | 5 | 6 | 7 | 8 | 9 |
| Part A | | | | | | | | | |
| Cyclomethicone | 16.10 | 25.90 | 12.00 | 16.20 | 18.99 | 11.7 | 15.00 | 26.99 | 16.30 |
| Dimethi-cone | 0 | 0 | 0.02 | 0 | 0.01 | 0 | 0 | 0.01 | 0 |
| copolyol | | | | | | | | | |
| Shin-Etsu | 0 | 0.1 | 0 | 0 | 0 | 0 | 0 | 0 | 0 |
| FPD-4668 | | | | | | | | | |
| Emulsogen SRO | 0.10 | 0 | 0 | 0 | 0 | 0 | 0 | 0 | 0 |
| PPG-3 myristyl | 3.00 | 3.00 | 0 | 3.00 | 0 | 2.5 | 2.00 | 2.00 | 2.80 |
| ether | | | | | | | | | |
| Silicone fluid | 0 | 0 | 16.98 | 0 | 20.00 | 0 | 0 | 0 | 0 |
| Hydrogenated | 0 | 0 | 0 | 0 | 0 | 0 | 2.20 | 0 | 0 |
| polyiso-butene 250 | | | | | | | | | |
| Fragrance | 0.80 | 1.00 | 1.00 | 0.80 | 1.00 | 0.80 | 0.80 | 1.00 | 0.80 |
| Part B | | | | | | | | | |
| AP active | 67.00 | 55.00 | 55.00 | 70.50 | 50.00 | 66 | 60 | 55.60 | 65.98 |
| (28% in water) | | | | | | | | | |
| Propylene glycol | 4.00 | 0 | 0 | 5.00 | 0 | 0 | 0 | 0 | 9.70 |
| Tripropylene glycol | 0 | 0 | 0 | 0 | 0 | 9 | 7 | 0 | 0 |
| Polymer JR | 0.20 | 0.20 | 0.20 | 0.20 | 0.20 | 0.30 | 0.25 | 0 | 0.40 |
| Alcohol (100%) | 8.80 | 9.80 | 9.80 | 4.30 | 4.80 | 5 | 9 | 9.00 | 0 |
| PureGel 990 | 0 | 0 | 0 | 0 | 0 | 0 | 0 | 2.40 | 0 |
| Additional Water | 0 | 5.00 | 5.00 | 0 | 5.00 | 4.70 | 3.75 | 3.00 | 4.00 |
| Oleath-10 | 0 | 0 | 0 | 0 | 0 | 0 | 0 | 0 | 0.02 |
| Total | 100 | 100 | 100 | 100 | 100 | 100 | 100 | 100 | 100 |
| Total water content | 48.24 | 44.6 | 44.6 | 50.8 | 41 | 52.2 | 46.9 | 43.03 | 51.5 |
| Oil:Water | 20:80 | 30:70 | 30:70 | 20:80 | 40:60 | 15:85 | 20:80 | 30:70 | 20:80 |

What is claimed is:

1. A two-phase, elastomer-free, low viscosity, high water roll-on antiperspirant and/or deodorant composition comprising:
(A) a non-polar phase having a viscosity up to 200 centipoise and comprising:
(a) 0.1-40 weight % of a volatile and/or a nonvolatile silicone selected from the group consisting of linear and cyclic organo-substituted polysiloxanes wherein the viscosity is less than 5 centistokes for volatile silicones and in the range of 5-20 centistokes for non-volatile silicones;
(b) 0-25 weight % of a straight or branched chain hydrocarbon polymer which has an average molecular weight in the range of 450-6000 daltons;
(c) 0-15 weight % of one or more low viscosity, lipophilic emollients selected from the group consisting of:
(i) 0-10 weight % of a non-polar hydrocarbon having from 4-30 carbons;
(ii) 0-10 weight % of a benzoic acid ester selected from the group consisting of C12-C20 benzoate esters;
(iii) 0-8 weight % of a propoxylated fatty alcohol having 4-16 carbons and 2-14 moles of propoxylation;
(d) optionally one or more ingredients selected from the group consisting of
(i) 0-0.2 weight % of a silicone surfactant having an hydrophilic lipophilic balance ≦13;
(ii) 0-2 weight % hydrophobically treated amorphous colloidal fumed silica;
(iii) 0-2 weight % fragrance;
(iv) 0-1.00 weight % vitamins; and
(v) 0.0-0.002 weight % coloring agent; and
(B) a polar phase having a viscosity in the range of 10-2,000 centipoise and comprising:
(a) at least 5 weight % of an antiperspirant active;
(b) an aqueous component comprising at least 40% water and a sufficient amount of a C2-3 alcohol, a glycol or a polyhydric alcohol so that the antiperspirant active is dissolved in the aqueous component;
(c) a thickening agent selected from the group consisting of:
(i) 0.05-2.5 weight % of a water soluble cationic derivative selected from the group consisting of hydroxyethyl cellulose and its copolymers and hydroxypropyl cellulose and its copolymers, provided that the viscosity of the polar phase does not exceed 2,000 cps; and
(ii) 0.1-5 weight % of a starch modified polymer which is a sodium hydroxypropyl starch phosphate;
(d) optionally one or more ingredients selected from the group consisting of
(i) 0-1.0 weight % mica with an average particle size in the range of 10-125;
(ii) an effective amount of a suspending agent;
(iii) an effective amount of an antimicrobial agent;
(iv) 0-5.0 weight % masking agent;
(v) 0-1.0 weight % of a polymer of ethylene oxide; and
(vi) 0-0.5 weight % of a water soluble surfactant having an hydrophilic lipophilic balance (“HLB value”)≧10;
wherein the ratio of oil phase to water phase is in the range of 15:85-40:60; and
whereby the composition is able to form a temporarily stabilized emulsion after shaking for a period not exceeding 24 hours.

2. A composition according to claim 1 which is free of clay.

3. A composition according to claim 1 which is free of clay and is free of silica.

4. A composition according to claim 1 wherein the period does not exceed 15 hours.

5. A composition according to claim 1 wherein the period is in the range of 1-20 minutes.

6. A composition according to claim 1 comprising 5-30 weight % of a linear or cyclic organo-substituted polysiloxane having a viscosity less than 20 centistokes.

7. A composition according to claim 1 comprising 1-10 weight % of the hydrocarbon polymer.

8. A composition according to claim 1 or 7 comprising 1-10 weight % of the lipophilic emollients.

9. A composition according to claim 1 comprising 1-10 weight % of the lipophilic emollients wherein the lipophilic emollients are selected from the group consisting of: (i) 1-8 weight % of the non-polar hydrocarbon; (ii) 1-8 weight % of the benzoic acid esters; and (iii) 4-6 weight % of the propoxylated fatty alcohol.

10. A composition according to claim 1 further comprising 0.001-0.01 weight % of the silicone surfactant.

11. A composition according to claim 1 further comprising 0.1-1 weight % of the hydrophobically treated amorphous colloidal fumed silica.

12. A composition according to claim 1 comprising 40-70 weight % water and up to 20 weight % of one or more members selected from the group consisting of ethyl alcohol, isopropyl alcohol, a glycol selected from the group consisting of propylene glycol, dipropylene glycol, tripropylene glycol and methylpropylene glycol, and mixtures of any of the foregoing.

13. A composition according to claim 1 comprising:
10-27 weight % pseudo silicone as a mixture of mineral oil, dodecanoic acid, dodecyl ester, hexadecanoic acid, octadecyl ester, docosanoic acid octadecyl ester;
1-2 weight % cyclomethicone;
1-3 weight % polybutene;
1-10 weight % PPG-3 myristyl ether;
45-58 weight % of Al/Zr tetrachlorohydrex gly as 30% in water or its equivalent;
0.1-0.5 weight % Polyquaternium-10;
2-10 weight % additional;
optionally 1-10 weight % ethanol; and optionally 0.5-1.0 weight % fragrance.

14. A composition according to claim 1 comprising:
0.5-1 weight % hydrophobically modified amorphous colloidal silica;
2-20 weight % cyclomethicone;
10-28 weight % dimethicone having a viscosity of 20 cst;
45-58 weight % of Al Zr tetrachlorohydrex gly as 30% in water or its equivalent;
0.1-0.5 weight % Polyquaternium-10;
2-10 weight % additional water;
optionally 1-10 weight % ethanol; and
optionally 0.5-1.0 weight % fragrance.

15. A composition according to claim 1 comprising:
10-20 weight % cyclomethicone;
0.05-0.5 weight % nonionic surfactant;
50-70 weight % antiperspirant active as 29% active in water or its equivalent;
10-15 weight % alcohol/glycol mixture as 1-10 % anhydrous ethanol+0-10% propylene glycol;
1-10 weight % additional water;
0.3-1 weight % Polyquaternium-10;
0.5-1.0 weight % fragrance; and
0-5 weight % emollient.

16. A composition according to claim 1 comprising:
20-30 weight % cyclomethicone;
0.05-0.2 weight % fluorinated silicone surfactant;
50-60 weight % antiperspirant active as 29% active in water or its equivalent;
5-15 weight % alcohol/glycol mixture as 1-10% anhydrous ethanol+0-10% propylene glycol;
1-10 weight % additional water;
0.1-0.3 weight % Polyquaternium-10;
0.5-1.0 weight % fragrance; and
0-5 weight % emollient.

17. A composition according to claim 1 comprising:
20-35 weight % cyclomethicone/dimethicone as a mixture of 10-35% D5 cyclomethicone+0-10% of dimethicone with a viscosity of <5 cst;
0.01-0.05 weight % silicone surfactant;
50-60 weight % antiperspirant active as 29% active in water or its equivalent;
5-15 weight % alcohol/glycol mixture as 1-10% anhydrous ethanol+0-10% propylene glycol;
1-10 weight % additional water;
0.1-0.3 weight % Polyquaternium-10;
0.5-1.0 weight % fragrance; and
0-5 weight % emollient.

18. A composition according to claim 1 comprising:
10-15 weight % cyclomethicone/dimethicone as a mixture of 5-15% D5 cyclomethicone+0-10% of dimethicone with a viscosity of <5 cst;
0.01-0.05 weight % silicone surfactant;
60-70 weight % as 29% active in water or its equivalent;
10-20 weight % alcohol/glycol mixture as 2-10% anhydrous ethanol+0-10% propylene glycol;
1-10 weight % additional water;
0.1-0.5 weight % Polyquaternium-10;
0.5-1.0 weight % fragrance; and
0-5 weight % emollient.

19. A composition according to claim 1 comprising:
10-20 weight % cyclomethicone/dimethicone as a mixture of 5-10% D5 cyclomethicone+0-10% of dimethicone with a viscosity of <5 cst;
50-70 weight % as 29% active in water or its equivalent;
10-20 weight % alcohol/glycol mixture as 2-10% anhydrous ethanol+0-10% propylene glycol;
1-10 weight % additional water;
0.1-0.5 weight % thickener;
0.5-1.0 weight % fragrance; and
1-10 weight % emollient.

20. A composition according to claim 1 comprising:
10-30 weight % cyclomethicone/dimethicone as a mixture of 10-30% D5 cyclomethicone+0-10% of dimethicone with a viscosity of <5 cst;
0.05-0.2 weight % silicone surfactant;
50-60 weight % as 29% active in water or its equivalent;
5-15 weight % alcohol/glycol mixture as 0-10% anhydrous ethanol+3-10% propylene glycol;
1-10 weight % additional water;
1-4 weight % thickener;
0.5-1.5 weight % fragrance; and
0-5 weight % emollient.
